(19) United States
(12) Patent Application Publication
(10) Pub. No.: US 2011/0098506 A1
(43) Pub. Date: Apr. 28, 2011

(54) METHOD OF PREPARING O-DESMETHYLVENLAFAXINE

(75) Inventors: Balaram Jivraj DHOTRE, Ahmedabad (IN); Ashesh Kamalnayan PANDYA, Ahmedabad (IN); Chiragkumar Anilkumar BHATT, Ahmedabad (IN); Chirag Girish NAIK, Ahmedabad (IN)

(73) Assignee: INTAS PHARMACEUTICALS LIMITED, Ahmedabad (IN)

(21) Appl. No.: 12/911,422

(22) Filed: Oct. 25, 2010

(30) Foreign Application Priority Data
Oct. 26, 2009 (IN) 2473/MUM/2009

(51) Int. Cl. C07C 213/10; C07C 213/00

(52) U.S. Cl. 564/336

(57) ABSTRACT

A method of preparing O-desmethylvenlafaxine from Venlafaxine or its pharmaceutically acceptable salt is disclosed. The disclosed method involves the use of mercapto carboxylic acids in the presence of suitable solvents under alkaline conditions. The disclosed process provides a novel and industrially feasible process for the preparation of O-desmethylvenlafaxine. The disclosed process also provides a cost effective, non-hazardous and efficient process for preparing O-desmethylvenlafaxine.

### FIELD

The present disclosure relates to a process for the preparation of O-desmethylvenlafaxine.

### BACKGROUND

O-desmethylvenlafaxine chemically named 1-[2-(dimethylamino)-1-(4-phenol)ethyl]-cyclohexanol was exemplified as a fumarate salt in U.S. Pat. No. 4,535,186 has the following formula (I).

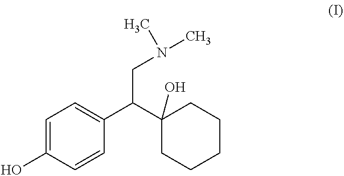

Klamerus, K. J. et al. (J. Clin. Pharmacol. 32:716-724), mentions that O-Desmethylvenlafaxine is a major metabolite of venlafaxine and has been shown to inhibit norepinephrine and serotonin uptake.

U.S. Pat. No. 6,689,912 describes a process for preparation of O-desmethylvenlafaxine, where venlafaxine is treated with a high molecular weight alkane or arene thiolate anion in an alcohol, ethylene glycol, ether of ethylene glycol or mixture.

U.S. Pat. No. 7,026,508 describes a process for preparation of O-desmethylvenlafaxine, which involves demethylating venlafaxine or a salt thereof with an alkali metal salt of a trialkylborohydride.

PCT Patent Publication No. WO 00/59851 describes a process for preparation of O-desmethylvenlafaxine, which involves contacting venlafaxine with lithium diphenylphosphide for a time and at a temperature sufficient to form O-desmethylvenlafaxine.

PCT Patent Publication No. WO 2007/071404 describes a process for preparation of O-desmethylvenlafaxine, which comprises combining metal sulfide, venlafaxine, and optionally selenium in a solvent and heating it sufficiently to obtain O-desmethylvenlafaxine.

PCT Patent Publication No. WO 2007/120923 describes a process for preparation of O-desmethylvenlafaxine, which comprises combining venlafaxine, an organic solvent and a reagent selected from the group consisting of thiophenol, sodium sulfide and a C1-C8 alkyl thiolate, heating the mixture and recovering O-desmethylvenlafaxine.

The above processes involve use of hazardous, toxic, costly and highly difficult-to-use reagents, which are not desirable on production scale. Also the yield and purity appear to be relatively low.

There is a need for cost effective, non hazardous and efficient process for preparing O-desmethylvenlafaxine, particularly those suitable for use on industrial scale.

### SUMMARY

A novel process for the preparation of O-desmethylvenlafaxine from Venlafaxine or its pharmaceutically acceptable salt is disclosed. The disclosed process involves the use of mercapto carboxylic acids in the presence of suitable solvents under alkaline conditions. The disclosed process provides a novel and industrially feasible process for the preparation of O-desmethylvenlafaxine. The disclosed process further provides a cost effective, non-hazardous and efficient process for preparing O-desmethylvenlafaxine.

### DETAILED DESCRIPTION

The present process for preparation of O-desmethylvenlafaxine of formula (I) involves demethylation of venlafaxine (II) or it's salt in presence of mercapto carboxylic acid and can be depicted by following scheme:

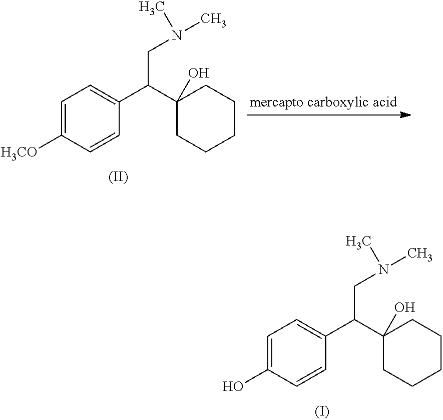

The mercapto carboxylic acids used as demethylating agent in present process can be selected from thioglycolic acid, 3-mercaptopropionic acid, thiomaleic acid, 4-mercapto benzoic acid, 4-mercapto phenylacetic acid or cysteine.

The salt of venlafaxine used in the disclosed process can be any salt, preferably, hydrochloride salt.

The above reaction is carried out in presence of suitable solvent and base. The solvent can be any organic solvent but preferably are selected from polar solvents such as dimethyl formamide, dimethyl acetamide or N-methylpyrrolidone or mixture thereof. Preferably strong base such as sodium hydroxide or potassium hydroxide is used in present process.

The O-demethylation of the present disclosure is carried out at high temperature; the reaction temperature may range from 100° C. to the reflux temperature of the solvent. However, the preferred temperature ranges from 120° to 165° C. The duration of reaction may vary form 10-25 hrs, more specifically 15-24 hrs. The reaction can be monitored by HPLC.

The O-desmethylvenlafaxine free base of formula (II) can be isolated from reaction mixture by following process:

i. adjusting the pH of reaction mixture to about 8-10;

ii. isolating desmethylvenlafaxine free base.

For isolation of O-desmethylvenlafaxine the pH of highly alkaline reaction mixture is adjusted towards lower range i.e. lower than 13. The product can be isolated at pH in range of 8-10, preferably product is isolated at pH 9.5.

Alternatively, O-desmethylvenlafaxine can be isolated from the reaction mixture by a process comprising steps of:

i. adjusting the pH of reaction mixture to about 5.0;

ii. increasing the pH to about 8-10;

iii. isolating desmethylvenlafaxine free base.

Any organic or inorganic acid can be used for pH adjustment, preferably hydrochloric acid is used.

The demethylating reagents used in present disclosure are safe to handle on large scale. Major advantage is the high boiling range of present reagents, which enables to carryout reaction at high temperature without loss of the reagents. This reduction in loss of demethylating reagents eventually leads to better yield.

Further advantage is presence of carboxylic group in demethylating agents which facilitate its removal in the basic condition after completion of reaction this simplifies the work up and also allows an odorless workup.

As a result we are able to obtain O-desmethylvenlafaxine having HPLC purity of about 99.5% and any single impurity is not more than 0.1%. If desired the product can be further recrystallised/purified by any organic solvent. The solvent used in the present process is methanol.

The O-desmethylvenlafaxine thus obtained can be converted to its pharmaceutically acceptable salts by methods known in the art.

Venlafaxine or its salts employed in present disclosure can be prepared by process disclosed in U.S. Pat. No. 4,535,186, which is herein incorporated by reference.

### EXAMPLES

The process of the present disclosure is in the following illustrated by examples that should not be construed to limit the scope of the disclosure in any manner:

### Example 1

### Preparation of O-Desmethylvenlafaxine Free Base by Using Thioglycolic Acid

A 1.0 litre, 4 neck flask equipped with thermometer and mechanical stirrer was charged with 50 g of Venlafaxine hydrochloride and 250 ml N-methylpyrrolidone. Then, 45.93 g NaOH was added to it. 55.09 g thioglycolic acid (80%) was added within 40-45 minutes. The temperature of reaction was raised 155-160° C. and it was stirred at the same for 15-20 hours. After completion of reaction the pH was adjusted to 9.5-10 with conc. HCl. The solid thus separated was filtered, purified by methanol and then dried to obtain 30 g of title compound.

HPLC purity—99.5%

### Example 2

### Preparation of O-Desmethylvenlafaxine Free Base by Using L-Cysteine

A 1.0 litre, 4 neck flask equipped with thermometer and mechanical stirrer was charged with 50 g of Venlafaxine hydrochloride and 250 ml N-methylpyrrolidone. Then, 45.93 g NaOH was added in it. 57.9 g L-Cysteine was added within 40-45 minutes. The temperature of reaction was raised to 155-160° C. and reaction mixture was stirred at the same for 15-20 hours. After completion of reaction the pH of reaction mixture was adjusted to 9-9.5 with conc. HCl. The product thus separated was filtered and then purified with methanol. The wet product was dried to obtain 28 g of title compound.

HPLC purity—99.6%

### Example 3

### Preparation of O-Desmethylvenlafaxine Free Base by Using Collic Acid

A 5.0 litre, 4 neck flask equipped with thermometer and mechanical stirrer was charged with the 500 g of Venlafaxine hydrochloride and 2.5 litre N-methyl pyrrolidone. Then, 458.8 g NaOH was added to it. 550.3 g thioglycolic acid (80%) was added within 40-45 minutes. Reaction was heated at 160-165° C. for 20-24 hours. The reaction was monitored by HPLC. The reaction was quenched with water and pH was adjusted to 5.5 to 6.0 with acetic acid to get clear solution. Activated charcoal was added, stirred for 30 minutes and filtered through celite bed. The pH of filtrate was adjusted 9.5-10 with aqueous NaOH solution. Separated solid was filtered and then purified by methanol. The wet product was filtered to obtain 302 g of title compound.

HPLC purity—99.5%

What is claimed is:

1. A process for preparation of O-desmethylvenlafaxine of formula (I) 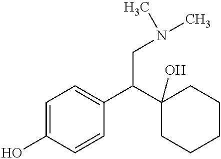
comprising O-demethylation of venlafaxine (II) 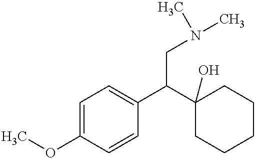
or its salt in presence of mercapto carboxylic acid.

2. The process according to claim 1, wherein the mercapto carboxylic acid is at least one selected from the group consisting of thioglycolic acid, 3-mercaptopropionic acid, thiomaleic acid, 4-mercapto benzoic acid, 4-mercapto phenylacetic acid and cysteine.

3. The process as claimed in claim 1, wherein O-demethylation of venlafaxine (II) or its salt comprises:
a. reacting venlafaxine (II) or its salt and mercapto carboxylic acid, in the presence of an organic solvent and a base;
b. isolating O-desmethylvenlafaxine (I) from the reaction mixture.

4. The process as claimed in claim 3, wherein the solvent selected from dimethyl formamide, N-methylpyrrolidone, N,N-dimethyl acetamide or mixture thereof.

5. The process according to claim 3, wherein the base is selected from sodium hydroxide or potassium hydroxide.

6. The process according to claim 3, wherein the O-demethylation is carried out at a temperature in range of 100-170° C.

7. The process according to claim 3, wherein isolation of O-desmethylvenlafaxine from reaction mixture comprises:
a. adjusting the pH of the reaction mixture in a. to about 8-10;
b. isolating O-desmethyl venlafaxine; and
c. optionally purifying O-desmethylvenlafaxine.

8. The process according to claim 3, wherein isolation of O-desmethylvenlafaxine comprises
a. adjusting the pH of the reaction mixture in a. to about 5;
b. adding the base to adjust the pH of the reaction mixture in a. to about 8-10;
c. isolating O-desmethyl venlafaxine; and
d. optionally purifying O-desmethylvenlafaxine

9. The process according to claim 7, wherein the pH of the reaction mixture is adjusted by using an acid.

10. The process as claimed in claim 9, wherein pH of reaction mixture is adjusted by using an acid selected from acetic acid and hydrochloric acid.

11. The process according to claim 8, wherein the pH of the reaction mixture is adjusted by using an acid.

12. The process as claimed in claim 11, wherein pH of reaction mixture is adjusted by using an acid selected from acetic acid and hydrochloric acid.

13. The process according to claim 8, wherein pH in step (b) is adjusted by using sodium hydroxide
